SEQUENCE LISTING (11 sequences)

SEQ ID NO: 1 - protein, 10 aa, synthetic construct
ACKHEWSGEC

SEQ ID NO: 2 - protein, 10 aa, synthetic construct
ACNPNPTSIC

SEQ ID NO: 3 - protein, 12 aa, synthetic construct
YAPSQTNPVVAT

SEQ ID NO: 4 - protein, 12 aa, synthetic construct
GIQSATAERTFR

SEQ ID NO: 5 - protein, 12 aa, synthetic construct
QRSIAHQQVLAL

SEQ ID NO: 6 - protein, 10 aa, synthetic construct
ACHANSSHYC

SEQ ID NO: 7 - protein, 12 aa, synthetic construct
QFDQGRLAYRSS

SEQ ID NO: 8 - protein, 12 aa, synthetic construct
SYTSPKERAVNL

SEQ ID NO: 9 - protein, 10 aa, synthetic construct
ACPGWDTRRC

SEQ ID NO: 10 - DNA, 20 bp, synthetic construct
  source 1..20: mol_type = other DNA
ccctcatagttagcgtaacg

SEQ ID NO: 11 - protein, 116 aa, Mus musculus
FTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPY

(19) United States
(12) Patent Application Publication
(10) Pub. No.: US 2026/0133194 A1
(43) Pub. Date: May 14, 2026

(54) PROGRAMMED CELL DEATH LIGAND 1 (PD-L1) TARGETING POLYPEPTIDE, MOLECULAR IMAGING PROBE, AND USE THEREOF

(71) Applicant: Peking Union Medical College Hospital, Chinese Academy of Medical Sciences, Beijing (CN)

(72) Inventors: Yining WANG, Beijing (CN); Yi Li, Beijing (CN)

(73) Assignee: Peking Union Medical College Hospital, Chinese Academy of Medical Sciences, Beijing (CN)

(21) Appl. No.: 19/361,121

(22) Filed: Oct. 17, 2025

(30) Foreign Application Priority Data
Nov. 8, 2024 (CN) 2024115897422

(51) Int. Cl. G01N 33/566; C07K 7/64

(52) U.S. Cl. CPC G01N 33/566; C07K 7/64; G01N 2800/24

(57) ABSTRACT

A programmed cell death ligand 1 (PD-L1) targeting polypeptide, a molecular imaging probe, and a use thereof are provided. The PD-L1 targeting polypeptide includes any one of the following polypeptides: (1) a polypeptide shown in at least one of SEQ ID NO: 1 to SEQ ID NO: 9; and (2) a polypeptide that is derived from an amino acid sequence of (1) through a substitution and/or a deletion and/or an addition of one or more amino acid residues and has the same function as the amino acid sequence. The magnetic resonance molecular imaging probe based on the PD-L1 targeting polypeptide enables the early diagnosis and evaluation of a tumor and an immune-mediated myocardial injury.

### CROSS REFERENCE TO THE RELATED APPLICATIONS

This application is based upon and claims priority to Chinese Patent Application No. 202411589742.2, filed on Nov. 8, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBBJHS015-PKGG_Sequence_Listing.xml, created on Oct. 17, 2025, and is 10,679 bytes in size.

### TECHNICAL FIELD

The present application relates to the technical field of biomedicine, and specifically relates to a programmed cell death ligand 1 (PD-L1) targeting polypeptide, a molecular imaging probe, and a use thereof.

### BACKGROUND

Immune checkpoint inhibitors (ICIs) have garnered widespread attention and research in comprehensive cancer therapy due to their ability to activate the immune system and provide specific targeting. ICIs can significantly improve the prognosis of cancer patients. Programmed cell death protein 1 (PD-1) is an inhibitory receptor on the surface of T cells. When binding to a ligand PD-L1 for PD-1, PD-1 can suppress the activity of T cell, thereby reducing the immune response. Many tumor cells evade attacks from the immune system by expressing PD-L1. PD-1/PD-L1 inhibitors can restore the anti-tumor function of T cells by blocking this pathway. With the increasing use of ICIs, the incidence of immune-related adverse events (irAEs) has also been gradually increasing. Relevant studies have shown that the incidence of severe irAEs in patients receiving combination immunotherapy is as high as 59%. Further, the occurrence of immunotherapy-related adverse cardiovascular events (including cardiovascular death, cardiac arrest, and high-grade atrioventricular block) has increased approximately four-fold. The mortality of immunotherapy-associated myocarditis ranges from 40% to 60%. Therefore, there is an urgent clinical need to develop technical approaches for early monitoring of immune-mediated cardiotoxic injury to guide treatment decisions.

### SUMMARY

Based on this, an embodiment of the present application provides a PD-L1 targeting polypeptide and constructs a magnetic resonance molecular imaging probe based on the PD-L1 targeting polypeptide to achieve the early diagnosis of an immune-mediated myocardial injury and guide treatment decisions based on the early monitoring of the immune-mediated myocardial injury.

Technical solutions are as follows:

An embodiment of the present application provides a PD-L1 targeting polypeptide, including any one of the following polypeptides:
(1) a polypeptide shown in at least one of SEQ ID NO: 1 to SEQ ID NO: 9; and
(2) a polypeptide that is derived from an amino acid sequence of (1) through a substitution and/or a deletion and/or an addition of one or more amino acid residues and has the same function as the amino acid sequence.

In a preferred embodiment, the PD-L1 targeting polypeptide includes a polypeptide shown in at least one of SEQ ID NO: 1 to SEQ ID NO: 4. This polypeptide exhibits high affinity. In a preferred embodiment, the PD-L1 targeting polypeptide includes a polypeptide shown in SEQ ID NO: 1.

An embodiment of the present application further provides a lipid carrier, including a targeting lipid including the PD-L1 targeting polypeptide.

In an embodiment, the targeting lipid further includes DSPE-PEG2000-NHS.

In an embodiment, the PD-L1 targeting polypeptide is linked to the DSPE-PEG2000-NHS through an amide bond.

In an embodiment, a molar ratio of the PD-L1 targeting polypeptide to the DSPE-PEG2000-NHS is 1:2.8.

In an embodiment, the PD-L1 targeting polypeptide is modified on a surface of the lipid carrier.

In an embodiment, the lipid carrier further includes an adjuvant.

In an embodiment, the adjuvant includes one or more of hydrogenated soybean phospholipid, cholesterol, and DSPE-PEG2000.

In an embodiment, in the lipid carrier, the hydrogenated soybean phospholipid, the cholesterol, the DSPE-PEG2000, and the targeting lipid are in a molar ratio of 55:40:3:2.

In an embodiment, the lipid carrier includes a lipid nanoparticle.

An embodiment of the present application further provides a molecular imaging probe, including the PD-L1 targeting polypeptide and further including a signal molecule.

The present application utilizes the phage display technology to select the PD-L1 targeting polypeptide, and constructs a molecular imaging probe based on the PD-L1 targeting polypeptide to provide a basis for the early diagnosis of an immune-mediated myocardial injury.

Lipid carriers are nanovesicles composed of phospholipid bilayers, and have recently gained significant attention as drug delivery vehicles. The passive targeting of target tissues can be achieved by modifying particle sizes of liposomes. Further, the lipid carrier can encapsulate a signal molecule (such as a magnetic resonance imaging contrast agent Gd-DTPA) to enable an imaging function.

Thus, an embodiment of the present application further provides a molecular imaging probe, including the lipid carrier and further including a signal molecule.

In an embodiment, the signal molecule is encapsulated within the lipid carrier.

In an embodiment, the signal molecule possesses an imaging function.

In an embodiment, the imaging function includes at least one of fluorescence imaging, magnetic resonance imaging, nuclear imaging, ultrasound imaging, photoacoustic imaging, and computed tomography imaging.

In an embodiment, a signal molecule with a magnetic resonance imaging function includes at least one of a gadolinium-based contrast agent (such as Gd-DTPA), an iron-based contrast agent, and a manganese-based contrast agent.

In an embodiment, the molecular imaging probe has an average particle size of 138.71±11.96 nm and an average charge of −10.60±1.51 mV.

An embodiment of the present application further provides a preparation method of the molecular imaging probe, including the following S1 to S3:
S1: Allowing the DSPE-PEG2000-NHS to undergo a reaction with the PD-L1 targeting polypeptide to produce the targeting lipid.

In an embodiment, the DSPE-PEG2000-NHS and the PD-L1 targeting polypeptide are dissolved in a dimethyl sulfoxide (DMSO) solution to allow the reaction.

In an embodiment, the reaction is conducted at room temperature for 1 h to 2 h.

In an embodiment, after the reaction, lyophilization is further included.

In an embodiment, the DSPE-PEG2000-NHS and the PD-L1 targeting polypeptide are dissolved in DMSO, incubated at room temperature for 1 h, and lyophilized to produce the targeting lipid.
S2: Dissolving the hydrogenated soybean phospholipid, the cholesterol, the DSPE-PEG2000, the targeting lipid, and the signal molecule in an organic solvent, thoroughly mixing, and removing the organic solvent to produce a lipid film.

In an embodiment, the organic solvent includes ethanol.

In an embodiment, an approach for the thoroughly mixing includes stirring.

In an embodiment, the ethanol is removed through rotary evaporation under vacuum.

In an embodiment, a mass-to-mole ratio of the targeting lipid to the signal molecule is (1-1.3) mg: 2 mmol. Optionally, the mass-to-mole ratio of the targeting lipid to the signal molecule is 1 mg: 2 mmol, 1.1 mg: 2 mmol, 1.2 mg: 2 mmol, 1.3 mg: 2 mmol, or any range formed by the aforementioned ratio values.
S3: Hydrating the lipid film to produce the molecular imaging probe.

In an embodiment, a hydration medium is added to the lipid film, rotary hydration is conducted for 50 min to 70 min, and an ultrasonic treatment is conducted to produce the molecular imaging probe.

In an embodiment, the hydration medium includes phosphate buffered saline (PBS).

In an embodiment, the ultrasonic treatment is conducted for 1 min to 2 min.

In an embodiment, after the ultrasonic treatment, filtration and lyophilization are further included.

An embodiment of the present application further provides a use of the PD-L1 targeting polypeptide, the lipid carrier, or the molecular imaging probe in preparation of a reagent for qualitative and/or quantitative detection of expression or expression localization of PD-L1.

An embodiment of the present application further provides a use of the PD-L1 targeting polypeptide, the lipid carrier, or the molecular imaging probe in preparation of a reagent for diagnosis or tracer imaging of a tumor or an immune-mediated myocardial injury.

The reagent for the tracer imaging is provided to monitor the progression of a disease associated with high PD-L1 expression, thereby providing a reference basis for clinical treatment decisions.

In an embodiment, the diagnosis includes early diagnosis of the tumor or the immune-mediated myocardial injury.

An embodiment of the present application further provides a use of the PD-L1 targeting polypeptide, the lipid carrier, or the molecular imaging probe in preparation of a drug for preventing and/or treating a tumor.

An embodiment of the present application further provides a use of the PD-L1 targeting polypeptide, the lipid carrier, or the molecular imaging probe in preparation of a product for assessing an abnormal PD-L1 expression-associated disease.

In an embodiment, the abnormal PD-L1 expression-associated disease includes an immune-mediated myocardial injury and a malignant tumor.

This assessment product can evaluate an extent of the immune-mediated myocardial injury or an extent of a progression and treatment of the tumor to guide clinical treatment decisions.

In an embodiment, abnormal PD-L1 expression includes abnormal elevation or abnormal reduction.

In an embodiment, the malignant tumor includes, but is not limited to, melanoma, lung cancer, and renal cell carcinoma.

The present application utilizes the phage display technology to select a PD-L1 targeting polypeptide, constructs a lipid carrier to encapsulate a magnetic resonance imaging contrast agent (Gd-DTPA), and modifies a surface of the lipid carrier with the PD-L1 targeting polypeptide to produce a molecular imaging probe. Due to the targetability of the PD-L1 polypeptide, the molecular imaging probe acquires a function of actively targeting an inflammatory site, and thus can achieve the early and accurate diagnosis of an immune-mediated myocardial injury.

Therefore, an embodiment of the present application further provides a product for early diagnosis or assessment of an immune-mediated myocardial injury, including the PD-L1 targeting polypeptide, the lipid carrier, or the molecular imaging probe.

In an embodiment, the product includes an early diagnosis or assessment kit.

Compared to the conventional techniques, the present application offers the following beneficial effects:

The present application employs the phage display technology to select and provide a cyclic and linear polypeptide specifically targeting PD-L1 and a sequence thereof. The polypeptide can be processed and modified, and thus can be subsequently used in the development of PD-L1-targeted probes and specifically-targeted drugs. Consequently, the polypeptide offers potential targeted diagnostic and therapeutic values for diseases associated with high PD-L1 expression.

Further, the present application provides a molecular imaging probe with a specific diagnostic function by constructing a lipid carrier modified with the PD-L1 targeting polypeptide on a surface and then encapsulating a contrast agent with an imaging function such as Gd-DTPA within the lipid carrier. The molecular imaging probe can actively deliver Gd-DTPA to a site of an immune-mediated myocardial injury in a targeted manner due to the specific targeting capability of the PD-L1 targeting polypeptide, thereby enabling the evaluation of the immune-mediated myocardial injury through magnetic resonance imaging. The molecular imaging probe exhibits excellent stability and a high drug-loading capacity. The preparation method of the molecular imaging probe is simple and controllable, can achieve the large-scale production, and holds a promising clinical application prospect.

### BRIEF DESCRIPTION OF THE DRAWINGS

To illustrate the technical solutions in the embodiments of the present application clearly and understand the present application and beneficial effects thereof comprehensively, the accompanying drawings required for describing the embodiments will be briefly described below. Apparently, the accompanying drawings in the following description show merely some embodiments of the present application, and other accompanying drawings may be derived from these accompanying drawings by those skilled in the art without creative efforts.

FIG. 1 shows phage enzyme linked immunosorbant assay (ELISA) evaluation results for affinities of PD-L1 targeting polypeptides to PD-L1;

FIGS. 2A-2X show Surface Plasmon Resonance (SPR) evaluation results for affinities of PD-L1 targeting polypeptides to PD-L1; where, FIGS. 2A-2F show affinity assessment of the PD-L1 targeting polypeptide PDP #1 at different concentrations of PD-L1;

FIGS. 2G-2L show affinity assessment of the PD-L1 targeting polypeptide PDP #2 at different concentrations of PD-L1; FIGS. 2M-2R show affinity assessment of the PD-L1 targeting polypeptide PDP #3 at different concentrations of PD-L1; and FIGS. 2S-2X show affinity assessment of the PD-L1 targeting polypeptide PDP #4 at different concentrations of PD-L1;

FIGS. 3A-3D show structural formulas of PD-L1 targeting polypeptides;

FIG. 4 shows structural superposition of a PD-L1 targeting polypeptide and a PD-L1 protein predicted by AlphaFold3;

FIG. 5 shows a binding mode of PDP #1 to a mouse PD-L1 protein, where the mouse PD-L1 protein is shown in green, PDP #1 is shown in blue, and interacting residues are represented as sticks; red dashed lines indicate hydrogen bonding interactions, and numbers indicate hydrogen bond lengths; and the larger the hydrogen bond length, the weaker the hydrogen bonding interaction;

FIG. 6 shows a transmission electron microscopy (TEM) image of a targeted molecular probe, which is a circular structure with a uniform particle size;

FIGS. 7A-7B show a particle size and charge of a targeted molecular probe;

FIG. 8 shows in vitro evaluation results for targeting efficacy of a targeted molecular probe against PD-L1; and

FIG. 9 shows in vivo evaluation results of a targeted molecular probe for an immune-mediated myocardial injury.

### DETAILED DESCRIPTION OF THE EMBODIMENTS

To make the above objectives, features, and advantages of the present application clearly understood, the specific implementations of the present application will be described in detail below. Many details are set forth in the following description to facilitate the comprehensive understanding of the present application. However, the present application may be implemented in many other ways other than those described herein, and those skilled in the art may make similar improvements without departing from the connotation of the present application. Therefore, the present application is not limited to the specific embodiments disclosed below.

Unless otherwise defined, all technical and scientific terms used herein have the same meanings as commonly understood by those skilled in the technical field of the present application. The terms used in the specification of the present application are merely for the purpose of describing specific embodiments, and are not intended to limit the present application.

The term “and/or” used herein includes any and all combinations of one or more related items listed.

Studies have shown that the expression of PD-L1 is significantly increased in myocardial tissues affected by immune-mediated myocarditis. Through the immunohistochemical staining of myocardial samples from clinical patients with immune-mediated myocardial injuries, the inventors of the present application have discovered significantly-elevated PD-L1 expression on surfaces of cardiomyocytes and endothelial cells in these myocardial samples. By establishing an immune-mediated myocarditis mouse model, the present application has found that the expression of PD-L1 by endothelial cells and cardiomyocytes of a myocardial tissue at an early stage of an immune-mediated myocardial injury increases. Therefore, the monitoring of PD-L1 expression in a myocardial tissue is expected to enable the early identification of an immune-mediated myocardial injury and provide a methodological basis for clinical decision-making.

The phage display technology is a powerful screening tool widely used for screening and identifying polypeptides and proteins with specific binding properties. Genes encoding the polypeptides or proteins are cloned into structural genes for phage coat proteins, such that the exogenous polypeptides or proteins undergo fusion expression with the coat proteins, and the expressed fusion proteins are displayed on surfaces of phages with the reassembly of the phages. The displayed polypeptides or proteins can retain relatively-independent spatial structures and biological activities, which facilitates the recognition and binding of target molecules. Through multiple rounds of screening procedures such as adsorption, elution, and amplification, phages with a specific affinity for a target are enriched. Through DNA sequencing for phages, a nucleic acid sequence encoding a polypeptide capable of binding to the target can be identified. In the present application, a polypeptide with specific targetability for PD-L1 is selected by the phage display technology, and the specific polypeptide is used for in vivo evaluation of PD-L1 expression.

Imaging examination methods based on molecular probes can dynamically monitor molecular-level changes in pathological tissues and cells, and have the potential of achieving the early diagnosis of immune-mediated myocardial injuries. 68Ga-DOTATOC-PET/CT is a highly-sensitive molecular probe for myocarditis, and can detect early myocardial injury lesions in cases where myocardial enzyme levels are elevated but magnetic resonance imaging results are normal. Due to complex operations, a low soft tissue resolution, and a specific radiation injury of PET/CT, the application of PET/CT to the diagnosis of myocardial injuries is restricted. Cardiac magnetic resonance is characterized by no radiation and a high soft tissue resolution. In the present application, the phage display technology is used to select a polypeptide sequence specifically targeting PD-L1, and a magnetic resonance molecular imaging probe based on the PD-L1 targeting polypeptide is constructed to enable the early assessment of an immune-mediated myocardial injury and provide a basis for clinical decision-making.

The present application provides a cyclic and linear polypeptide targeting PD-L1 selected by the phage display technology, and a use thereof in diagnosis of an immune-mediated myocardial injury.

PD-L1 is an inhibitory immune checkpoint molecule widely expressed on surfaces of cardiomyocytes and endothelial cells when there is an immune-mediated myocardial injury. A polypeptide-based molecular imaging probe targeting PD-L1 is constructed to assess an extent of a myocardial injury associated with immune-mediated myocarditis and guide clinical treatment decisions.

The present application provides a method for selecting a cyclic and linear polypeptide targeting PD-L1 based on the phage display technology, including the following steps: A PD-L1 protein is subjected to multiple rounds of incubation and panning with a phage display library. Phages binding to PD-L1 are recovered and amplified, and a coding sequence for the polypeptide is acquired through sequencing. The selected cyclic and linear polypeptide is synthesized, and an affinity of the cyclic and linear polypeptide to PD-L1 is verified through in vitro binding assay. An in vitro cell experiment is conducted for the selected polypeptide to evaluate a binding capacity of the selected polypeptide to PD-L1. A diagnostic efficacy of a molecular imaging probe based on the PD-L1 targeting polypeptide is assessed in a myocardial injury animal model.

The polypeptide selected by the aforementioned method in the present application exhibits high affinity and specificity, and can effectively bind to PD-L1. In the in vitro experiment, the polypeptide exhibits strong affinity for PD-L1. The molecular imaging probe based on the polypeptide demonstrates a prominent imaging effect for evaluating an extent of a myocardial injury in the myocardial injury animal model. Therefore, the polypeptide selected in the present application holds a promising application prospect for the early assessment of an immune-mediated myocardial injury. The present application provides a PD-L1 targeting polypeptide sequence and a use thereof in diagnosis of an immune-mediated myocardial injury, which are expected to provide an approach for efficient and safe assessment of an immune-mediated myocardial injury.

The embodiments of the present application will be described in detail below with reference to examples. It should be understood that these examples are merely intended to describe the present application rather than limit the scope of the present application. In the following examples, the experimental methods without specified conditions are preferentially implemented with reference to the guidelines in the present application, may be implemented with reference to laboratory manuals or conventional conditions in the art, may be implemented with reference to conditions recommended by manufacturers, or may be implemented with reference to the experimental methods known in the art.

In the following specific examples, measurement parameters for raw material components may undergo minor deviations within weighing accuracy ranges, unless otherwise specified. For temperature and time parameters, an acceptable deviation resulting from a measuring accuracy or an operational accuracy of an instrument is allowed.

The phage display peptide library used in the following examples is commercially obtained from a biotechnology company.

The polypeptides specifically targeting PD-L1 adopted in the following examples are synthesized by a biotechnology company, and have a purity of greater than 95%. Before an experiment, a targeting polypeptide used is prepared into a stock solution with an appropriate concentration using a solvent.

The PD-L1 targeting peptides in the following examples all can be prepared through standard Fmoc solid-phase synthesis.

### Example 1 Screening of PD-L1 Targeting Polypeptides by the Phage Display Technology

### 1. Method for Detecting a Phage Titer

(1) An ER2738 strain was inoculated into an LB medium (including 30 μg/mL of a Tet antibiotic) for activation, then inoculated at a ratio of 1:100 into 5 mL of a fresh LB medium (including 30 μg/mL of the Tet antibiotic), and cultured until OD₆₀₀ was 0.4 to 0.6.
(2) Preparation of agar plates: 1.25 g of isopropyl beta-D-thiogalactopyranoside (IPTG) and 1 g of X-gal were dissolved in 25 mL of N,N-dimethylformamide (DMF) to produce an IPTG/X-gal stock solution, and the IPTG/X-gal stock solution was stored at −20° C. for later use. 15 g of agar and 1 mL of the IPTG/X-gal stock solution were added per 1 L of an LB medium to prepare the agar plates. The agar plates were stored at 4° C. in the dark for later use.
(3) A phage was diluted with an LB medium by a 10-fold serial dilution method. A host strain culture resulting from the expanded culture was equally divided into tubes as required, and 10 μL of a serially diluted phage suspension was added to each tube. Resulting mixtures were gently mixed and incubated at room temperature for 30 min.
(4) The infected host strain obtained in the step (3) was coated on the agar plates prepared in the step (2), and cultured overnight at 37° C.
(5) Plaque counting was conducted.

### 2. Expanded Culture of Phages

(1) An ER2738 strain was inoculated into an LB medium (including 30 μg/mL of a Tet antibiotic) for activation, then inoculated at a ratio of 1:100 into 5 mL of a fresh LB medium (including 30 μg/mL of the Tet antibiotic), and cultured until OD₆₀₀ was 0.4 to 0.6.
(2) 10 μL of a phage was inoculated into a host strain culture resulting from the expanded culture, and cultured for 3 h to 6 h under shaking at 200 rpm and 37° C. Centrifugation was conducted at 8,000 rpm, and a resulting supernatant was collected, which was a phage culture.

### 3. Screening of a Phage Display Peptide Library

(1) Antigen coating: A PD-L1 protein standard was diluted with PBS to 5 μg/mL. A 96-well ELISA plate was taken, and two replicate wells were set for each condition. 100 μL of a PD-L1 protein standard dilution was added to each well (400 ng/well), and coating was conducted overnight at 4° C. PBS was adopted as a negative control.
(2) Blocking: The coating solution was discarded, 200 μL of a 2% skimmed milk powder was added to each well, and blocking was conducted at room temperature for 1 h.
(3) Phage incubation: The ELISA plate was washed three times with PBST. A phage solution prepared in the step 1 was taken, diluted with a 2% skimmed milk powder to 5×10¹¹ pfu/mL, and added at 200 μL/well to the ELISA plate, and the ELISA plate was then incubated at room temperature for 2 h.
(4) Elution: A phage sample was discarded, and the ELISA plate was washed three times with PBST. 200 μL of Tris-HCl (pH=3.0) was added to each well, and the ELISA plate was allowed to stand at room temperature for 10 min. A resulting eluate was collected and promptly neutralized with Tris-HCl at 40 μL/well (pH=8.0) to approximately pH=7.0 (an amount of Tris-HCl at pH=8.0 should be re-determined prior to each neutralization).
(5) Determination of a phage titer in the eluate: The phage titer in the eluate was determined according to the method in the step 1.
(6) The expanded culture and screening steps were repeated twice to complete the second and third rounds of panning.
(7) Positive phage clones were sequenced using a sequencing primer −96gIII (5′-CCCTCATAGTTAGCGTAACG-3′, SEQ ID NO: 10) in a reverse direction.
(8) 36 gene sequences located between two enzyme cleavage sites were identified, and translated into amino acid sequences on the web.expasy.org/translate/website. Translation results of 3′-5′ were polypeptide sequences displayed by phages, as shown in Table 1.

TABLE 1
PD-L1 targeting polypeptide sequences selected by
the phage display technology

| No. | Peptide Sequence |
|---|---|
| PDP#1 | ACKHEWSGEC, SEQ ID NO: 1 |
| PDP#2 | ACNPNPTSIC, SEQ ID NO: 2 |
| PDP#3 | YAPSQTNPVVAT, SEQ ID NO: 3 |
| PDP#4 | GIQSATAERTFR, SEQ ID NO: 4 |
| PDP#5 | QRSIAHQQVLAL, SEQ ID NO: 5 |
| PDP#6 | ACHANSSHYC, SEQ ID NO: 6 |
| PDP#7 | QFDQGRLAYRSS, SEQ ID NO: 7 |
| PDP#8 | SYTSPKERAVNL, SEQ ID NO: 8 |
| PDP#9 | ACPGWDTRRC, SEQ ID NO: 9 |

### Example 2 Detection of Affinities of the PD-L1 Targeting Polypeptides

### 1. Phage ELISA

(1) An ELISA plate was coated with a PD-L1 protein and incubated at 4° C. overnight.
(2) The ELISA plate was blocked with a 0.5% (w/v) bovine serum albumin (BSA) solution for 1 h.
(3) The ELISA plate was washed three times with 0.1% TBST. Monoclonal phages (100 μL, about 1.0×10¹¹ pfu/mL, diluted in TBS) were added to each well, and the ELISA plate was then incubated for 2 h under shaking at room temperature. TBS was adopted as a control.
(4) A horseradish peroxidase (HRP)-conjugated anti-M13 monoclonal antibody (1:5,000) was added to the ELISA plate, and the ELISA plate was incubated at room temperature for 1 h and then washed three times with 0.1% TBST.
(5) A reaction buffer and a stop buffer were added.
(6) The absorbance at 405 nm was measured using an ultraviolet spectrophotometer.

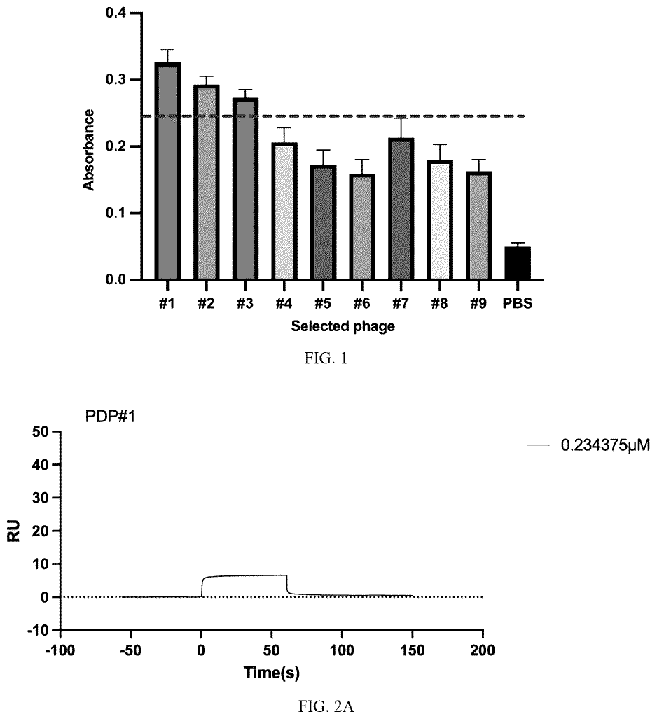

A selected and synthesized polypeptide capable of binding to PD-L1 was subjected to an in vitro binding test with the coated PD-L1 protein. Test results were shown in FIG. 1. It could be seen that all polypeptides could bind to the PD-L1 protein in vitro, and the absorbance after binding was detected at 405 nm for each polypeptide.

### 2. Surface-Plasmon Resonance (SPR)

### 2.1 Protein Conjugation

(1) A running buffer (200 mL of 1×PBS), a water bottle, and a waste liquid bottle were placed on left and right trays, respectively, and the corresponding liquid inlet tubes were inserted.
(2) A CM5 chip was hand-held with a printed side facing upward, and gently inserted into a slot following a direction indicated by an arrow on the chip. Finally, a door of a chip compartment was closed.
(3) Channel 2 of the chip was activated with 1-ethyl-3-(3-dimethylaminopropyl) carbodiimide (EDC, GE Healthcare) and N-hydroxysuccinimide (NHS, GE Healthcare) at a flow rate of 10 μL/min.
(4) A ligand protein was diluted with a sodium acetate buffer at pH 4.0 to 50 μg/mL, and immobilized on the channel 2 of the chip at a flow rate of 10 μL/min. A conjugation level was set to 9,000, and a conjugation sensorgram was generated.
(5) The channel was blocked with ethanolamine at a flow rate of 10 μL/min.
(6) The steps (3) to (5) were repeated for reference channel 1, except that, in the step (4), a protein-free acetate buffer was adopted instead.

### 2.2 Solvent Correction

1. PBS-P+ including 5% of DMSO was adopted as a running buffer for an analyte. The original running buffer on the left tray of the system was replaced with the 1.PBS-P+ including 5% of DMSO, and the corresponding liquid inlet tube was inserted. 5% DMSO solutions were prepared by mixing 4.5% and 5.8% stock solutions according to Table 2 to determine a calibration curve.

TABLE 2
Formulas for solvent correction solutions

| Buffer/Vial | 1 | 2 | 3 | 4 | 5 | 6 | 7 | 8 |
|---|---|---|---|---|---|---|---|---|
| 4.5% | 0 | 200 | 400 | 600 | 800 | 1000 | 1200 | 1400 |
| DMSO(μL) | | | | | | | | |
| 5.8% | 1400 | 1200 | 1000 | 800 | 600 | 400 | 200 | 0 |
| DMSO(μL) | | | | | | | | |

### 2.3 Analyte Measurement

(1) Each analyte was diluted to several concentrations in a 96-well plate, and allowed to bind to a target protein through the chip from low to high concentrations, with a flow rate of 30 μL/min and a duration of 150 s.
(2) After an analyte solution at each concentration flowed through the chip, the chip was regenerated with a 10 mM glycine hydrochloride solution (pH 2.0) for 5 min. This process was repeated until all corresponding concentrations of an analyte had been tested.
(3) Data of a sample was acquired using BIAcore T200 Control software (v.2.0, GE Healthcare), and subtracted by data of the reference channel. Resulting data was globally fitted to a 1:1 Langmuir binding model using BIAcore T200 evaluation software to obtain binding and dissociation constants.

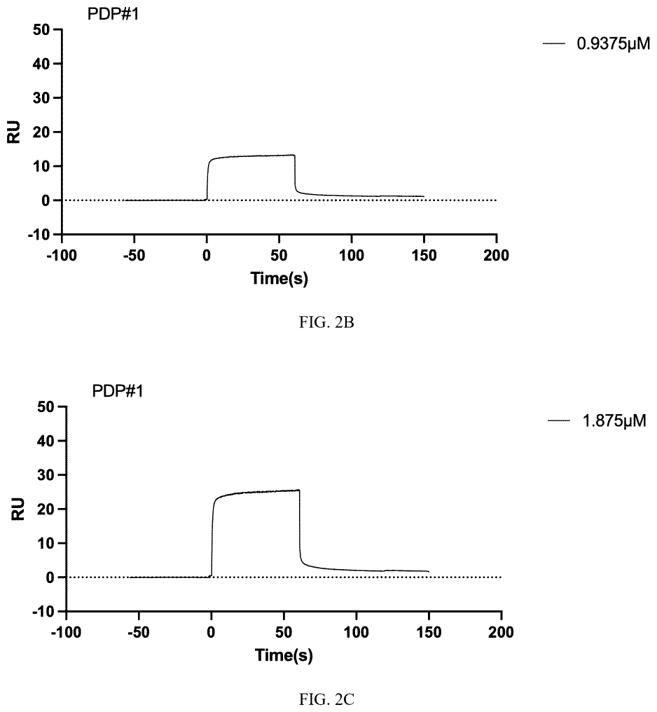

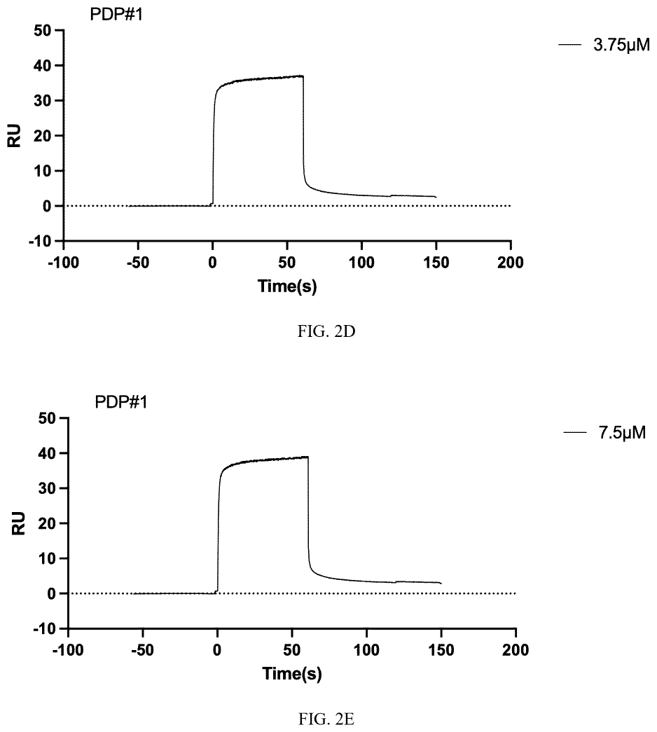

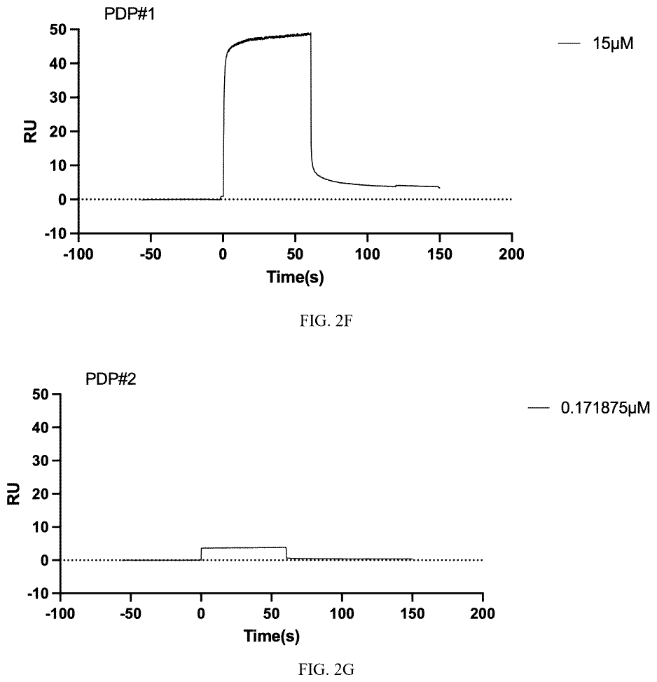

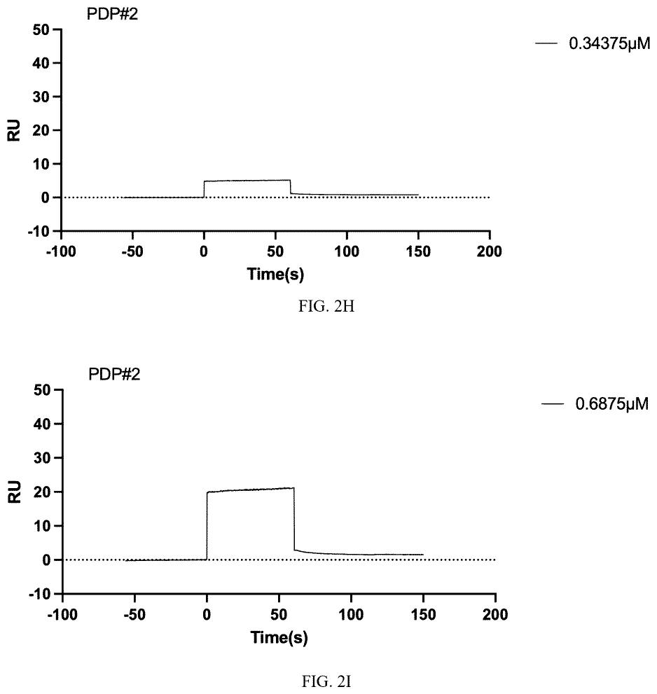

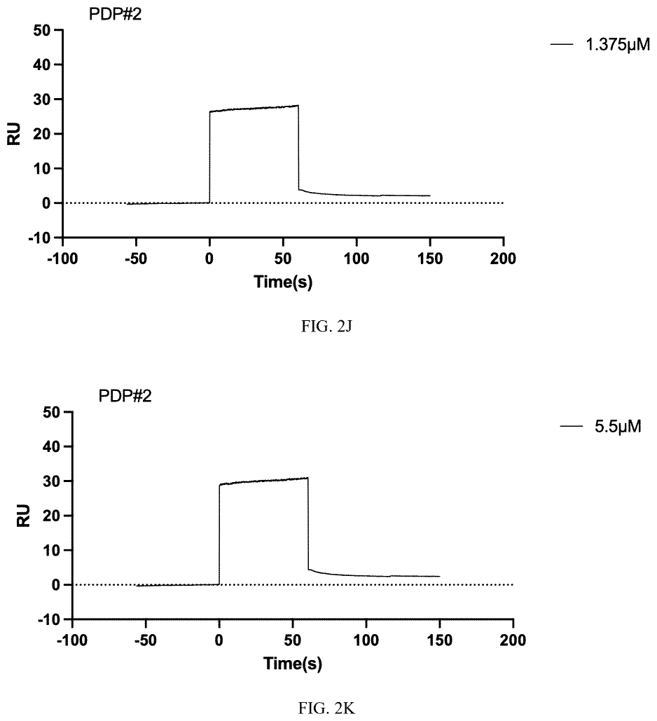

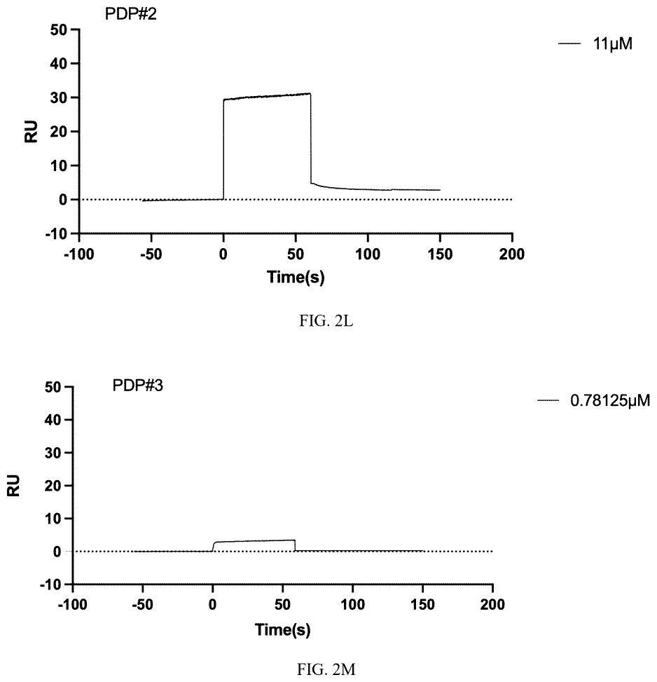

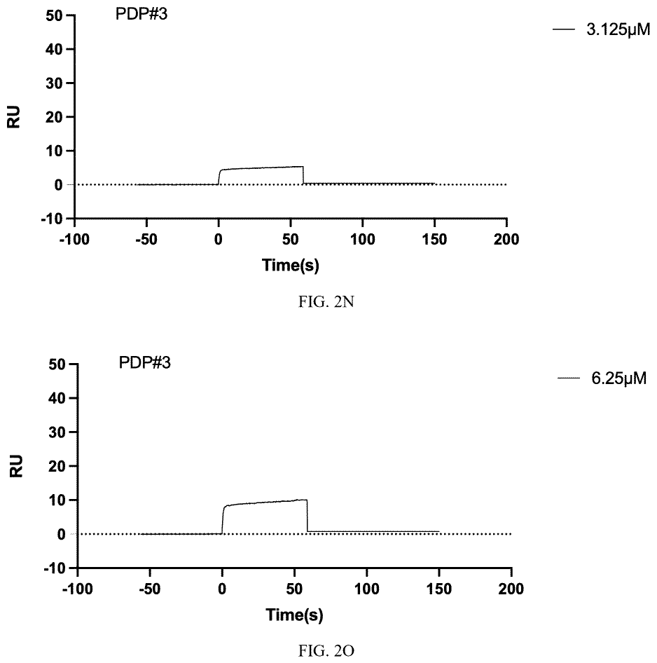

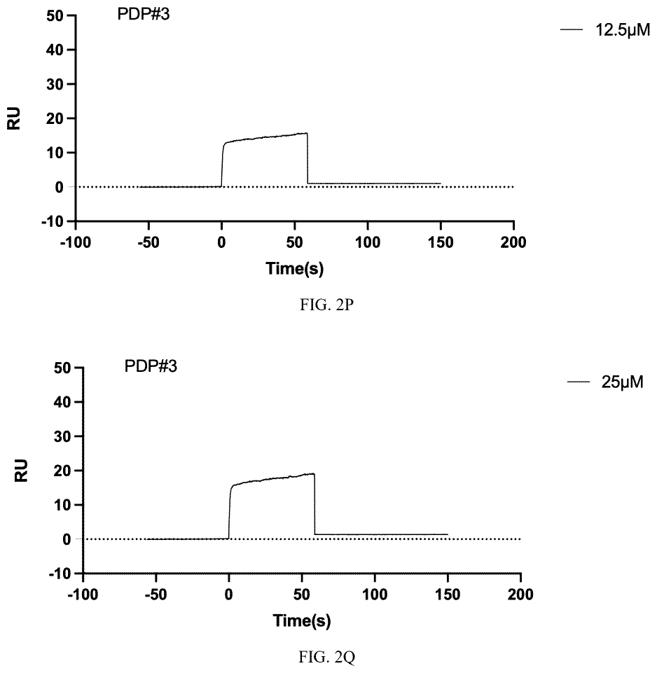

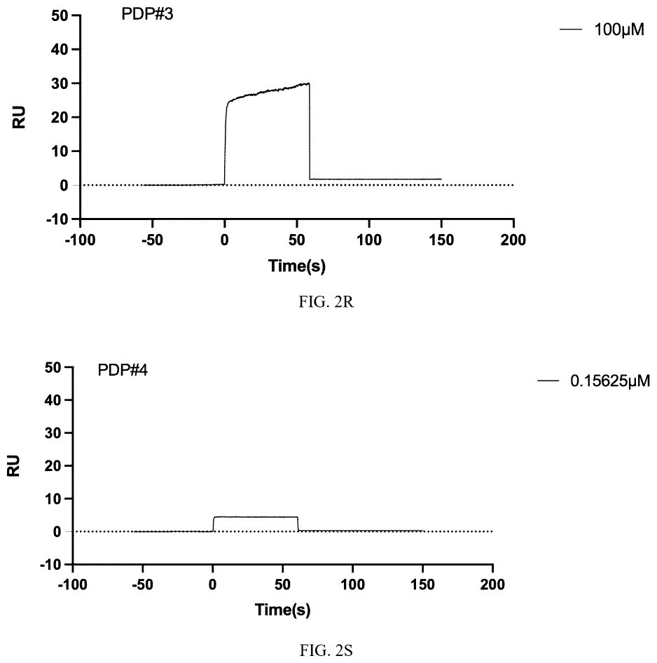

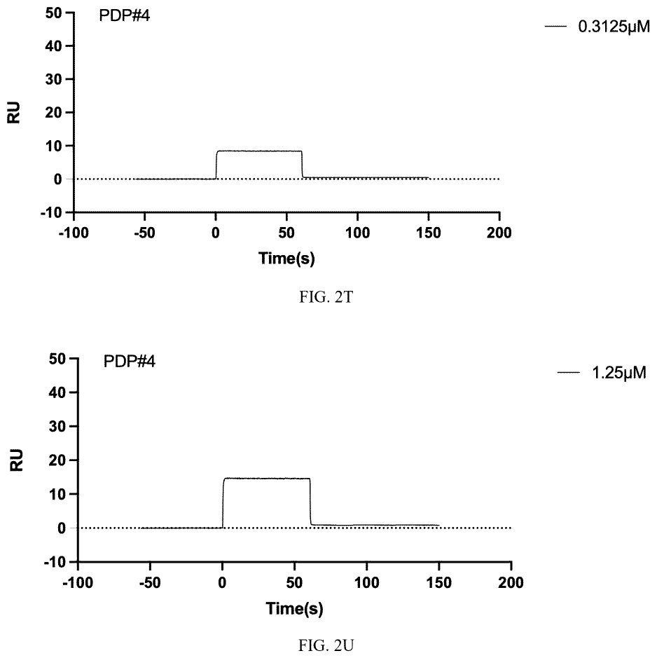

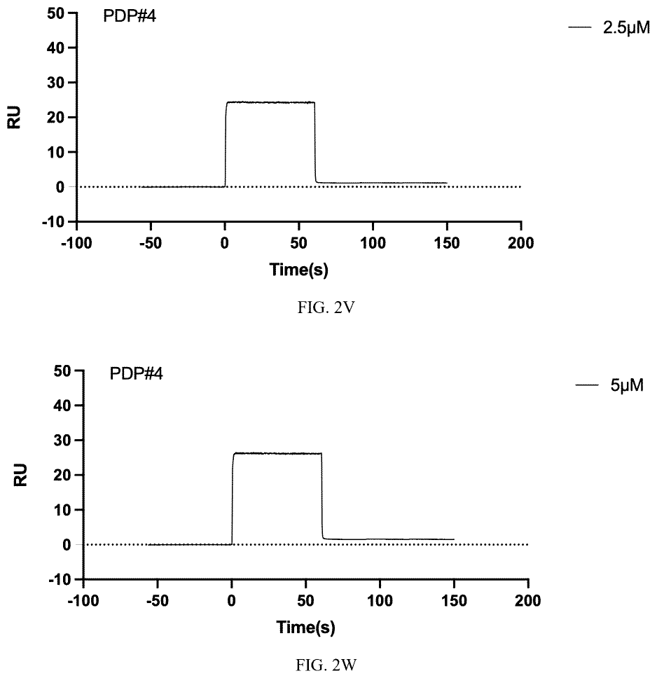

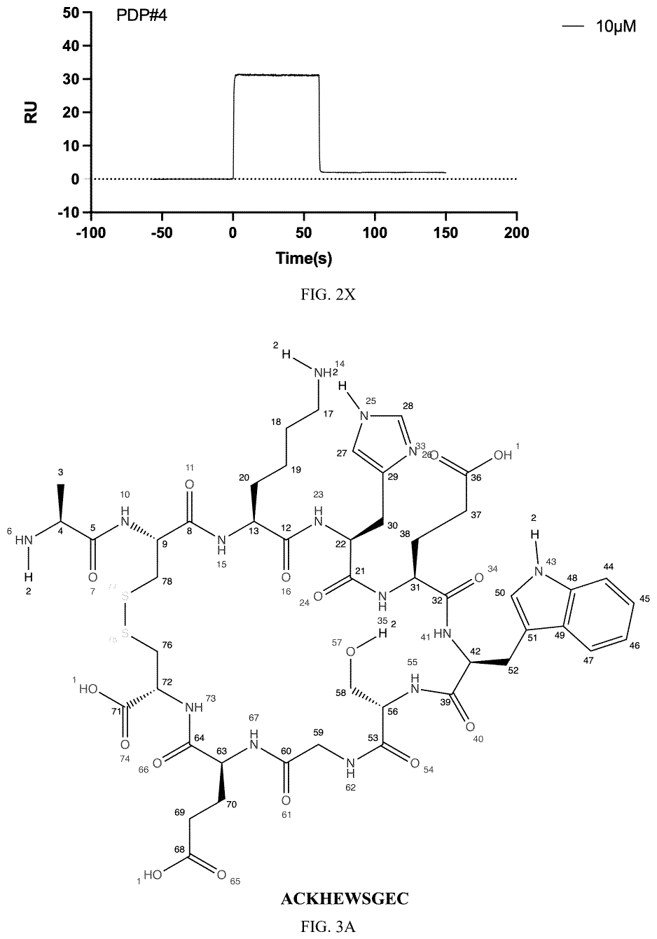

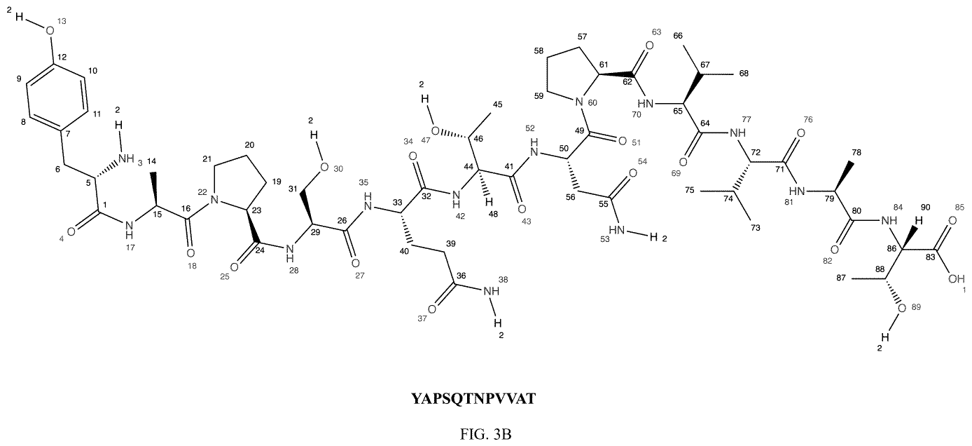

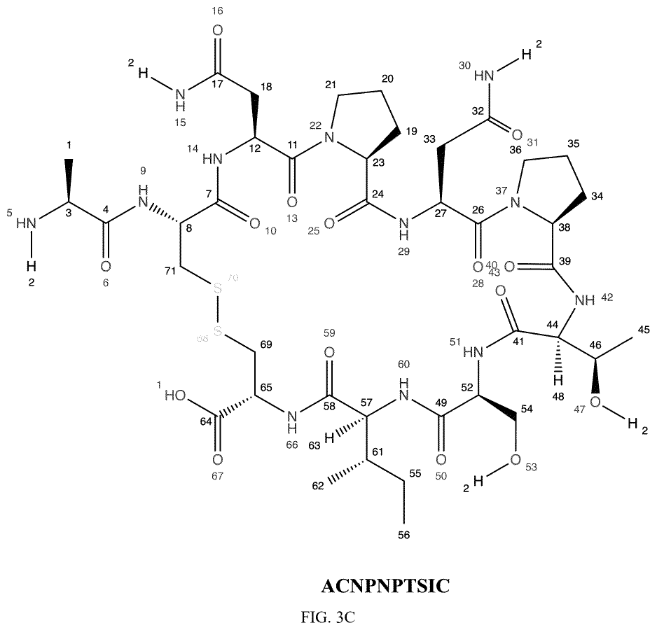

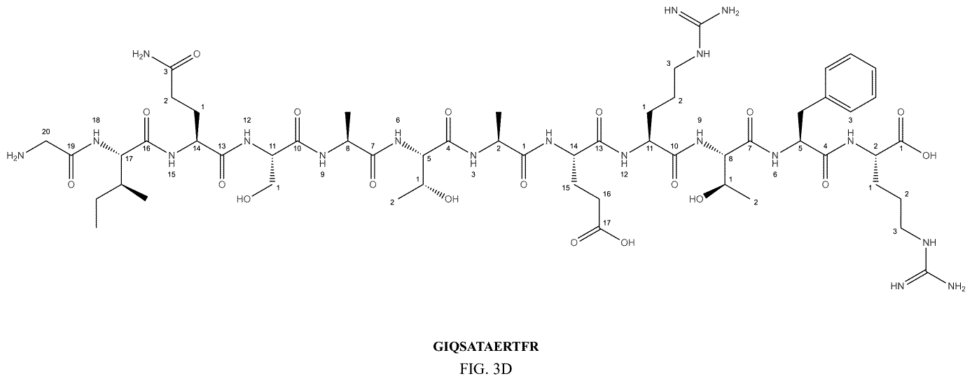

An affinity of a polypeptide to the PD-L1 protein was determined by SPR according to a dissolution concentration. Determination results were shown in Table 3. SPR results were shown in FIGS. 2A-2X. The targeting polypeptides exhibited high affinities for PD-L1, where pep1 demonstrated the strongest binding affinity for PD-L1. pep1 was a cyclic polypeptide, and structural formulas of this cyclic polypeptide were shown in FIGS. 3A-3D.

TABLE 3
SPR determination results of affinities
of the polypeptides for PD-L1

| Ligand | Polypeptide | KD(M) | Ka (1/Ms) | KD(1/S) |
|---|---|---|---|---|
| PDL1 | PDP#1 | 2.11E−6 | 1.55E+5 | 3.27E−1 |
| | PDP#2 | 7.23E−6 | 5.56E+4 | 4.02E−1 |
| | PDP#3 | 6.83E−5 | 9.51E+3 | 5.55E−1 |
| | PDP#4 | 1.43E−5 | 3.79E+4 | 5.53E−1 |

### 3. Molecular Docking

A sequence of the mouse PD-L1 protein was downloaded from the Uniprot website, and the following interval sequence corresponding to a PD-1 binding domain (6SRU: 19-134) was extracted:

Mouse PD-L1:
SEQ ID NO: 11
FTITAPKDLYVVEYGSNVTMECRFPVERELDLLALVVYWEKEDEQVIQF
VAGEEDLKPQHSNFRGRASLPKDQLLKGNAALQITDVKLQDAGVYCCII
SYGGADYKRITLKVNAPY,.

PDP #1: ACKHEWSGEC (SEQ ID NO: 1) was subjected to molecular docking with the mouse PD-L1.

In this project, AlphaFold3 Server was used for protein-polypeptide docking. The mouse PD-L1 was docked with the PDP #1 polypeptide, and the optimal conformation (model_0) was selected from docking results and analyzed.

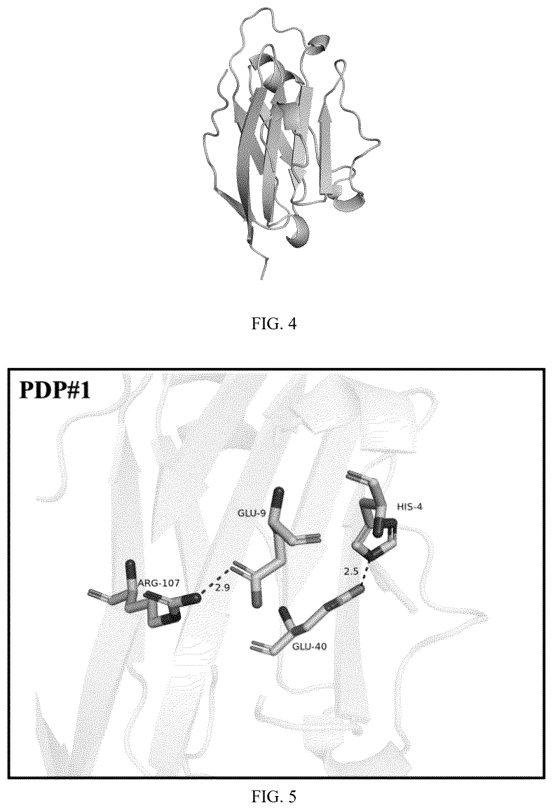

The three-dimensional structure 6SRU of the mouse PD-L1 analyzed by the experiment was essentially consistent with a structure predicted by AlphaFold3. A root-mean-square deviation (RMSD) after the superposition between the predicted and experimental structures was 0.299, as shown in FIG. 4. A docking mode between the PDP #1 polypeptide and the mouse PD-L1 protein was shown in FIG. 5.

### Example 3 Preparation and Characterization of a Molecular Imaging Probe Targeting PD-L1

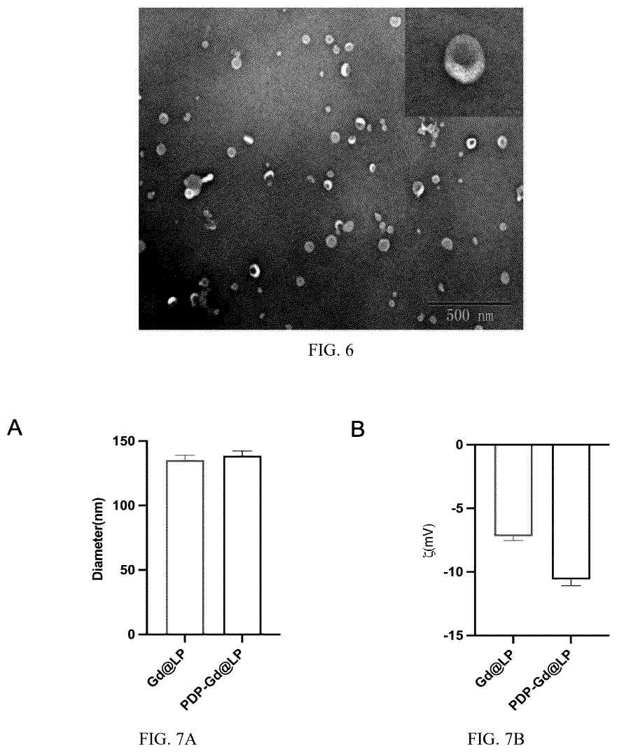

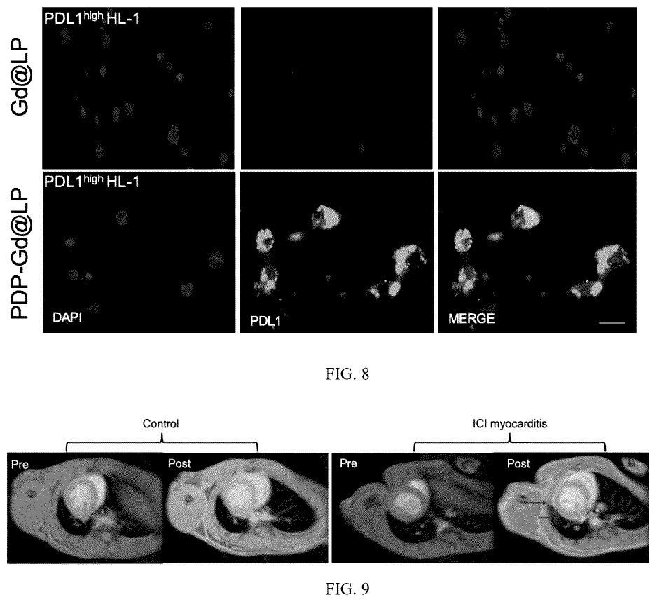

(1) Preparation of a polypeptide-lipid conjugate: A PD-L1 targeting polypeptide was synthesized through solid-phase synthesis (During the synthesis, an acetyl (AC) group was added at a head end and an amino (NH₂) group was added at a tail end to cap the ends of the polypeptide). The polypeptide sequence adopted for the preparation of the molecular imaging probe was as follows: PDP #1: AC-ACKHEWSGEC-NH₂ (SEQ ID NO: 1). A molar ratio of DSPE-PEG2000-NHS to the PD-L1 targeting polypeptide was 1:2.8. 6.37 mg of the DSPE-PEG2000-NHS and 7.18 mg of the PDP #1 were weighed and dissolved in DMSO, incubated at room temperature for 1 h, then lyophilized for later use.
(2) 27.99 mg of hydrogenated soybean phospholipid, 10.33 mg of cholesterol, 5.5 mg of DSPE-mPEG2000, 6.18 mg of the polypeptide-lipid conjugate prepared in the step (1), and 1 mL (100 mM) of a magnetic resonance imaging contrast agent were taken. The materials taken above were co-dissolved in 5 mL of an ethanol solution, stirred for 30 min, and then subjected to rotary evaporation under vacuum to remove ethanol. Then, 5 mL of PBS was added, and stirring was conducted for 5 min to allow hydration. Rotary evaporation was conducted with a rotary evaporator for 60 min. Subsequently, an ultrasonic treatment was conducted for 1 min in an ice-water bath with a probe-type ultrasonic instrument to produce a magnetic resonance imaging contrast agent-encapsulated liposome solution. The magnetic resonance imaging contrast agent-encapsulated liposome solution was ultrafiltered four times using an ultrafiltration tube with a pore size of 3.5 KD for 40 min each time to remove the unencapsulated magnetic resonance imaging contrast agent, so as to produce a magnetic resonance imaging contrast agent-encapsulated targeted liposome solution. The magnetic resonance imaging contrast agent-encapsulated targeted liposome solution was lyophilized for later use.
(3) The molecular imaging probe prepared in this example was subjected to morphological characterization by TEM. The molecular imaging probe presented as individually-dispersed nanoparticles with a regular morphology, as shown in FIG. 6.
(4) The molecular imaging probe prepared in this example was characterized for a particle size and a potential based on a nanoparticle size, a concentration, and a zeta potential analyzer. The molecular imaging probe prepared in this example had an average particle size of 138.71±11.96 nm and an average charge of −10.60±1.51 mV, as shown in FIGS. 7A-7B.
(5) In vitro PD-L1 targeting experiment for the molecular imaging probe: To validate the specific binding of the PD-L1 targeting probe to PD-L1, both targeting and non-targeting molecular probes were co-cultured with LV-PDL1-infected HL-1 cells (with high PD-L1 expression). PDL1^{high} HL-1 cells were inoculated into a 6-well plate at a density of 10⁵ cells/well and cultured for 24 h. A DIO-labeled targeting molecular imaging probe or a DIL-labeled non-targeting molecular imaging probe was added to a cell culture plate and incubated at 37° C. for 4 h. Then, washing was conducted three times with PBS to remove the unbound liposomes. Cells were fixed with 4% paraformaldehyde (PFA), and nuclei were stained with 4′,6-diamidino-2-phenylindole (DAPI) for 10 min. Fluorescence images were acquired using a confocal laser scanning microscope. The confocal imaging revealed that the molecular imaging probe underwent co-localization with the PD-L1 protein on the surface of HL-1 cells, indicating that the molecular imaging probe had a strong targeting ability for PD-L1, as shown in FIG. 8.
(6) In vivo evaluation of the molecular imaging probe for an immune-mediated myocardial injury: Female BALB/c mice aged 6 weeks to 8 weeks were selected. The combination of subcutaneous injection of a MyHc polypeptide and intraperitoneal injection of an anti-PD1 monoclonal antibody was adopted to establish an immune-mediated myocardial injury model. A control group was administered with equivalent amounts of a complete Freund's adjuvant and an IgG isotype control antibody. Model mice were divided into two groups, and the two groups were administered with 150 μL of either of the targeting and non-targeting molecular imaging probes through tail vein injection (150 μL per mouse). Cardiac magnetic resonance imaging was conducted 10 min after the administration. Experimental results were shown in FIG. 9. The non-targeting probe exhibited a poor cardiac targeting ability, but the targeting probe exhibited a prominent imaging effect for a myocardial injury.

The PD-L1 targeting polypeptide provided in the present application shows an affinity for the PD-L1 protein. The targeting molecular imaging probe prepared accordingly can be used for the assessment of a disease associated with high PD-L1 expression.

Through in vitro cellular-level experiments and immune-mediated myocardial injury mouse experiments, the present application proves that the PD-L1 targeting polypeptide exhibits significant targetability for PD-L1 both in vitro and in vivo, can be used for the preparation of a molecular imaging probe targeting PD-L1 and even an antitumor drug, and demonstrates a promising prospect for clinical transformation.

The technical characteristics of the above examples can be arbitrarily combined. For brevity of description, not all possible combinations of the technical characteristics of the above examples are described. However, these combinations of the technical characteristics should be construed as falling within the scope defined by the specification as long as there is no contradiction among the combinations.

The above examples only illustrate several embodiments of the present application, and the description thereof is specific and detailed, but cannot be construed as a limitation to the scope of the invention patent. It should be noted that those of ordinary skill in the art can further make several variations and improvements without departing from the concept of the present application, and these variations and improvements all fall within the protection scope of the present application. Therefore, the protection scope of the present application should be subject to the protection scope defined by the claims, and the specification may be used to interpret the content of the claims.

What is claimed is:

1. A programmed cell death ligand 1 (PD-L1) targeting polypeptide, wherein the PD-L1 targeting polypeptide is shown in SEQ ID NO: 1.

2. A lipid carrier, comprising a targeting lipid comprising the PD-L1 targeting polypeptide according to claim 1.

3. The lipid carrier according to claim 2, wherein the targeting lipid further comprises DSPE-PEG2000-NHS.

4. The lipid carrier according to claim 3, wherein the PD-L1 targeting polypeptide is linked to the DSPE-PEG2000-NHS through an amide bond.

5. The lipid carrier according to claim 4, wherein a molar ratio of the PD-L1 targeting polypeptide to the DSPE-PEG2000-NHS is 1:2.8.

6. The lipid carrier according to claim 2, wherein the PD-L1 targeting polypeptide is modified on a surface of the lipid carrier.

7. The lipid carrier according to claim 3, wherein the lipid carrier further comprises an adjuvant.

8. The lipid carrier according to claim 7, wherein the adjuvant comprises one or more of hydrogenated soybean phospholipid, cholesterol, and DSPE-PEG2000.

9. The lipid carrier according to claim 8, wherein in the lipid carrier, the hydrogenated soybean phospholipid, the cholesterol, the DSPE-PEG2000, and the targeting lipid are in a molar ratio of 55:40:3:2.

10. A molecular imaging probe, comprising the PD-L1 targeting polypeptide according to claim 1 or a lipid carrier and further comprising a signal molecule, wherein the lipid carrier comprises a targeting lipid comprising the PD-L1 targeting polypeptide.

11. The molecular imaging probe according to claim 10, wherein the signal molecule is encapsulated within the lipid carrier.

12. The molecular imaging probe according to claim 11, wherein the signal molecule possesses an imaging function.

13. The molecular imaging probe according to claim 12, wherein the imaging function comprises at least one of fluorescence imaging, magnetic resonance imaging, nuclear imaging, ultrasound imaging, photoacoustic imaging, and computed tomography imaging.

14. The molecular imaging probe according to claim 13, wherein a signal molecule with a magnetic resonance imaging function comprises at least one of a gadolinium-based contrast agent, an iron-based contrast agent, and a manganese-based contrast agent.

15. A preparation method of the molecular imaging probe according to claim 10, comprising preparing the molecular imaging probe by a film dispersion method.

16. The preparation method according to claim 15, comprising following steps:
allowing DSPE-PEG2000-NHS to undergo a reaction with the PD-L1 targeting polypeptide to produce the targeting lipid; and
dissolving hydrogenated soybean phospholipid, cholesterol, DSPE-PEG2000, the targeting lipid, and the signal molecule in an organic solvent, thoroughly mixing, and removing the organic solvent to produce a lipid film; and hydrating the lipid film to produce the molecular imaging probe.

17. The preparation method according to claim 16, wherein the preparation method satisfies one or more of following conditions:
(1) the DSPE-PEG2000-NHS and the PD-L1 targeting polypeptide are dissolved in a dimethyl sulfoxide (DMSO) solution to allow the reaction for 1 h to 2 h; and
(2) a mass-to-mole ratio of the targeting lipid to the signal molecule is (1-1.3) mg: 2 mmol.

18. A use of the PD-L1 targeting polypeptide according to claim 1, a lipid carrier, or a molecular imaging probe, comprising one or more of following aspects:
(1) a use in preparation of a reagent for diagnosis or tracer imaging of a tumor or an immune-mediated myocardial injury, wherein the tumor is melanoma, lung cancer, or renal cell carcinoma;
(2) a use in preparation of a drug for preventing and/or treating a tumor, wherein the tumor is melanoma, lung cancer, or renal cell carcinoma; and
(3) a use in preparation of a product for assessing an abnormally-high PD-L1 expression-associated disease,
wherein the abnormally-high PD-L1 expression-associated disease is selected from an immune-mediated myocardial injury and a tumor, wherein the tumor is melanoma, lung cancer, or renal cell carcinoma;
wherein the lipid carrier comprises a targeting lipid comprising the PD-L1 targeting polypeptide, and the molecular imaging probe comprises the PD-L1 targeting polypeptide or the lipid carrier and further comprises a signal molecule.

19. A product for early diagnosis or assessment of an immune-mediated myocardial injury, comprising the PD-L1 targeting polypeptide according to claim 1, a lipid carrier, or a molecular imaging probe, wherein the lipid carrier comprises a targeting lipid comprising the PD-L1 targeting polypeptide, and the molecular imaging probe comprises the PD-L1 targeting polypeptide or the lipid carrier and further comprises a signal molecule.

20. The lipid carrier according to claim 3, wherein the PD-L1 targeting polypeptide is modified on a surface of the lipid carrier.
